(19) United States
(12) Patent Application Publication
(10) Pub. No.: US 2019/0069866 A1
(43) Pub. Date: Mar. 7, 2019

(54) DISPLAY METHOD, AND DISPLAY CONTROL DEVICE

(71) Applicant: DENSO CORPORATION, Kariya-city, Aichi-pref. (JP)

(72) Inventor: Hideki OKUDA, Kariya-city, Aichi-pref. (JP)

(21) Appl. No.: 15/765,499

(22) PCT Filed: Oct. 7, 2016
(86) PCT No.: PCT/JP2016/080020

§ 371 Date: Apr. 3, 2018

(30) Foreign Application Priority Data
Oct. 7, 2015 (JP) 2015-199655

(51) Int. Cl. A61B 6/00; A61B 5/055; A61B 6/03; A61B 5/00

(52) U.S. Cl. CPC A61B 6/506; A61B 5/055; A61B 2017/00119; A61B 5/4041; A61B 6/03

(57) ABSTRACT

A display control device includes an image acquisition unit, a position acquisition unit, an image displaying unit, an information acquisition unit, and an information displaying unit. The image displaying unit displays with a display device a target image, the target image being an image a position corresponding to a tool position. The information acquisition unit acquires the at least one medical support information associated with an acquired position that represents the position of the medical tool at a time when the medical support information was acquired. The information displaying unit performs an information display by associating, based on the acquired position associated with the at least one medical support information, the medical support information with a position on the target image and that displays the medical support information.

### CROSS-REFERENCE TO RELATED APPLICATIONS

The present application is based on and claims priority from Japanese patent Application No. 2015-199655 filed on Oct. 7, 2015 with the Japanese Patent Office, the content of which is incorporated herein by reference.

### TECHNICAL FIELD

The present disclosure relates to a technique for displaying medical support information.

### BACKGROUND

Devices have been proposed for displaying, as medical support information necessary for supporting surgery as a medical practice, a tomographic image of an affected area of a patient and a neuromonitoring result in parallel (refer to Patent Literature 1).

### PRIOR ART LITERATURES

### Patent Literatures

Patent Literature 1: JP-2010-516400-A

### SUMMARY OF INVENTION

According to the device described in Patent Literature 1, a tomographic image and a neuromonitoring result are only displayed in parallel. As a result of detailed consideration by the present inventor, an issue was discovered in that when a practitioner views that display, it is difficult to directly recognize what kind of relationship exists between the tomographic image and the neuromonitoring result.

In other words, with conventional techniques, there is an issue in that it is difficult for a practitioner to recognize information necessary for medical practice.

Accordingly, among techniques for displaying medical support information, the present disclosure provides a technique for improving the recognition of medical support information by practitioners.

One aspect of the present disclosure is a display method for a display control device to display, with a display device, at least one medical support information related to supporting a medical practice. This display method includes acquiring modality images including at least one image capturing parts including an affected area of a patient, repeatedly acquiring a tool position, the tool position being a current position of a medical tool used in the medical practice, displaying with the display device a target image, the target image being the image among the acquired modality images at a position corresponding to the acquired tool position, and acquiring at least one medical support information. Here, at least one medical support information refers to medical support information which is associated with an acquired position that represents the position of the medical tool at a time when the medical support information was acquired.

Further, an information display is performed by associating, based on the acquired position associated with the acquired at least one medical support information, the medical support information with a position on the target image displayed by the display device and displaying the medical support information.

According to such a display method, at least one medical support information may be associated with a position on a target image and be displayed. For this reason, according to the display method, it may be easier for a practitioner to recognize the corresponding relationship between at least one medical support information and a target image.

Further, the target image displayed according to this display method is an image at a position corresponding to the tool position, the tool position being the current position of a medical tool. For this reason, according to the display method, it may be easier for a practitioner to recognize the corresponding relationship between at least one medical support information and the tool position.

As a result, according to the display method, it is possible to provide a technique for allowing practitioners to more easily recognize medical support information, in techniques which display medical support information.

As another aspect of the present disclosure, there is a display control device that displays with a display device at least one medical support information.

This display control device includes an image acquisition unit that acquires modality images, a position acquisition unit that repeatedly acquires a tool position, and an image displaying unit that displays with the display device a target image. Further, this display control device includes an information acquisition unit that acquires at least one medical support information, and an information displaying unit that performs an information display which associates the at least one medical support information with a position on the target image and displays the at least one medical support information.

According to such a display control device, the same effects as the above described display method may be exhibited.

Further, any reference numerals in parenthesis in the recitation of the claims are for the purpose of showing corresponding relationships between specific implementations described in the below embodiments in one example. These reference numerals do not limit the technical scope of the present disclosure.

### BRIEF DESCRIPTION OF DRAWINGS

FIG. 1 is a block diagram showing an outline configuration of a medical support system.

FIG. 2 is a flowchart showing processing steps of information registration processing.

FIG. 3 is a flowchart showing processing steps of information display processing.

FIG. 4 is an explanatory view showing an example of a display by information display processing.

FIG. 5 is an explanatory view showing an example of setting partition lines by information display processing.

FIG. 6 is an explanatory view showing a modified example of setting partition lines.

FIG. 7 is an explanatory view showing a modified example of setting partition lines.

FIG. 8 is an explanatory view showing a modified example of a display by information display processing.

### DESCRIPTION OF REFERENCE NUMERALS

1 . . . Medical Support System 3 . . . Imaging Device 5 . . . Storage Device 10 . . . Medical Navigation Device 12 . . . Position Identification Unit 14 . . . Registration Unit 20 . . . Device 30 . . . Display Control Device 32 . . . Control Unit 34 . . . ROM 36 . . . RAM 38 . . . CPU 40 . . . Timing Unit 42 . . . Storage Unit 44 . . . Input

Reception Unit 50 . . . Display 60 . . . Image Display Region 64 . . . Display Sphere 66, 67, 68 . . . Partition Line 70 . . . Information Display Frame 72 . . . Display Frame 74 . . . Lead Line

### Embodiments for Carrying out Invention

Embodiments of the present disclosure will be explained below with reference to the drawings.

(1-1. Medical Support System)

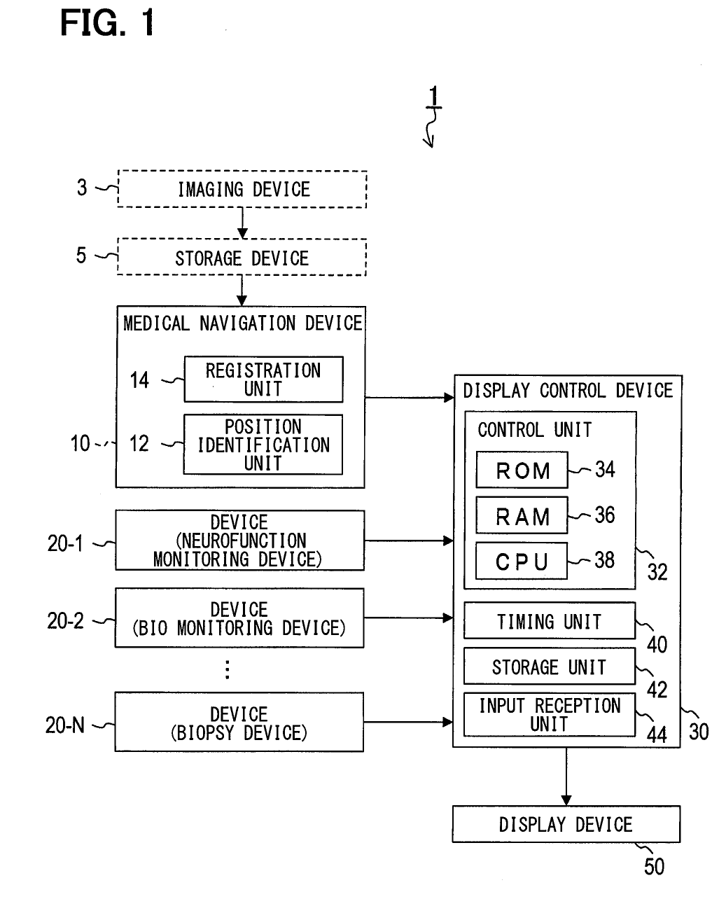

A medical support system 1 showing in FIG. 1 is a system that displays images obtained by capturing parts including an affected area of a patient, and that displays information related to supporting the medical practice of a practitioner with respect to the patient.

Here, the term medical practice includes surgical operations that involve incision of a patient to provide medical treatment. Here, the term surgical operation may refer to a variety of surgical operations such as brain surgery or heart surgery.

In the present embodiment, the images displayed by the medical support system 1 are modality images.

The modality images are images captured by an imaging device 3, which will be described below. The modality images are images which include at least one image capturing parts including an affected area of a patient. As an image that corresponds to these modality images, a three dimensional image of parts including an affected area of a patient is contemplated. The three dimensional image may, for example, be formed by a plurality of tomographic images captured from the affected part of the patient.

The imaging device 3 is a medical image diagnostic device. Here, medical image diagnostic devices include, for example, a nuclear magnetic resonance imaging device (so-called MRI), an X-ray imaging device, a medical ultrasonic examination device, a nuclear medicine diagnosis device (so-called PET examination apparatus), an endoscope apparatus, etc.

Further, the modality images captured by the imaging device 3 are stored in a storage device 5. Here, the storage device 5 refers to , for example, conventional storage devices with readable and writable memory contents.

The medical support system 1 includes a medical navigation device 10, at least one device 20, a display device 50, and a display control device 30.

The medical navigation device 10 is a conventional device for supporting medical practices of a practitioner, and includes a position identification unit 12 and a registration unit 14.

The position identification unit 12 identifies the current position in real space for a medical tool used in medical practice (hereinafter referred to as a tool position). Here, medical tools include surgical tools used in surgery. Here, surgical tools may include, for example, a scalpel, an electric scalpel, tweezers, forceps, a medical microscope, etc.

Here, the position identification unit 12 may identify the position of a medical tool by using conventional methods. For example, the position of a medical tool may be identified by placing a marker, which is prepared in advance, at a particular position on a medical tool, and then the position of the medical tool may be identified as a vector from a predetermined reference position to the particular position on the medical tool within a space where a medical procedure is performed. Further, such a vector may be identified by, for example, taking images of the market within the space where the medical procedure is performed, then performing image processing on those images.

The registration unit 14 associates coordinates of the modality images with coordinates for the space where the medical procedure is performed. This association of coordinates may be performed by conventional registration techniques of converting the coordinate system of the modality images to the coordinate system of the space where the medical procedure is performed.

The device 20 is a device used in medical practice. In the present embodiment, the device 20 may be a neural function monitoring device, a bio monitoring device, a bio inspection device, an air conditioner, etc. Further, the device 20 may be a medical navigation device, a computer tomography device, a nuclear magnetic resonance imaging device, an X-ray imaging device, a medical ultrasonic examination device, a nuclear medicine diagnosis device, an endoscope device, etc.

Here, a neural function monitoring device refers to a conventional device for detecting and monitoring the neural functions of a patient undergoing medical treatment.

Here, a bio monitoring device refers to a conventional device for monitoring the biological information of a patient undergoing medical treatment. Here, biological information refers to so-called vital signs. Here, vital signs include, for example, electrocardiogram, heart rate, blood pressure, body temperature, respiration, pulse, oxygen saturation, heart rate, brain wave, myoelectricity, anesthesia depth, motion induced potential, somatosensory induced potential, etc.

Here, a bio inspection device refers to conventional device for performing biological inspection with respect to systems (i.e., cells) of affected parts (e.g., lesion site) of a patient. Here, biological inspection refers to an inspection that diagnoses an illness or examines the degree of expansion of an illness by collecting and monitoring affected parts.

An air conditioner refers to a device for performing air conditioning of the space where the medical procedure is performed, and outputs data of temperature, humidity, air volume etc. for this space.

The display device 50 is a device for displaying information from the display control device 30. For example, a conventional liquid crystal display may be used as this display device 50.

The display control device 30 is a conventional controller and includes a control unit 32, a timing unit 40, a storage unit 42, and an input reception unit 44.

The control unit 32 is a conventional microcomputer including a ROM 34, a RAM 36, and a CPU 38. The ROM 34 stores data or programs which must be retained in memory even when power is turned off. The RAM 36 temporarily stores data. The CPU 38 performs processing by executing programs stored in the ROM 34 or the RAM 36.

The timing unit 40 measures an absolute time. The storage unit 42 is a conventional nonvolatile storage device configured with readable and writable storage contents.

The input reception unit 44 is a conventional input reception unit that receives input of information. This input reception unit 44 includes various types of input devices, for example, a keyboard or pointing device, switches, a microphone, etc. Here, a pointing device includes touchpads and touch panels. Here, a touchpad may be integrally formed with the display device 50.

Processing programs for the control unit 32 to perform an information registration processing are stored in the ROM 34 of the control unit 32. Here, the information registration processing refers to a processing where, when a piece of information from the device 20 satisfies a predetermined specified condition, that piece of information is treated as medical support information, associated with an acquired position with is the position of a medical tool at the time of the specified condition being satisfied, and stored in memory.

Further, processing programs for the control unit 32 to perform an information display processing are stored in the ROM 34 of the control unit 32. Here, the information display processing refers to a processing where modality images corresponding to the tool position are displayed and, together with this, medical support information associated with an acquired position within a region specified by that tool position is displayed.

(1-2. Information Registration Processing)

Next, an information registration processing performed by the control unit 32 will be explained.

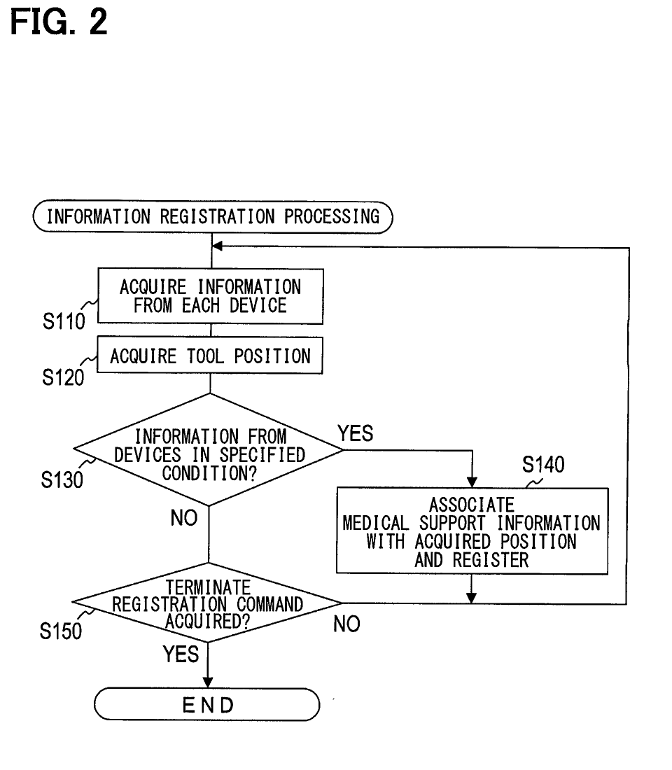

When this information registration processing is started, as shown in FIG. 2, the control unit 32 acquires information from each device 20 (S110). At S110, the control unit 32 acquires results of neural function monitoring, bio information, results of bio inspection, etc. from each device 20.

Next, in the information registration processing, the control unit 20 acquires a tool position identified by the position identification unit 12 of the medical navigation device 10 (S120). Further, the control unit 32 determines whether or not at least one of the information acquired from each device at S110 shows a predetermined specified condition (S130). Here, a specified condition refers to a predetermined specified condition, such as a threshold for prohibiting the continuation of a medical procedure by a practitioner, and is specified for each information from the devices 20.

At S130, for example, when a signal voltage potential representing the results of neural function monitoring indicates a predetermined specified condition, this information may be determined as representing a specified condition. Further, at S130, for example, when signal voltage potentials representing respective bio information indicates a predetermined specified condition, this information may be determined as representing a specified condition. Further, at S130, for example, when the result of bio inspection indicates a pathological change in the cells of an inspection target, this information may be determined as representing a specified condition.

When the result of the determination at S130 is that all information from each device 20 are not a specified condition (S130: NO), then the control unit 32 continues to S150 of the information registration processing, which is described later. Conversely, when the result of the determination at S130 is at least one piece of information from the devices 20 is a specified condition (S130:YES), the control unit 32 continues to S140 of the information registration processing.

At S140, the control unit 32 stores the information acquired from the device 20, which represents a specified condition, as medical support information in the storage unit 42. Here, medical support information refers to information that supports medical procedures by a practitioner.

Specifically, at S140, the control unit 32 associates the medical support information (i.e., the piece of medical support information) with an acquired position representing the position of a medical tool at the time when this medical support information was acquired (in other words, the tool position acquired at S120), and stores the medical support information in the storage unit 42. Further, when the inspection results from a bio inspection device is stored as the medical support information in the storage unit 42, the acquired information associated with this medical support information is the part of a patient from which pathological tissue is collected.

Further, at S140, the control unit 32 associates the medical support information with the absolute time at which point this medical support information was acquired, and stores the medical support information in the storage unit 42. Further, the absolute time associated with the medical support information may be measured by the timing unit 40.

Next, the control unit 32 returns to S110 of the information registration processing.

Meanwhile, at S150, which is performed when all information from each device 20 are not a specified condition, the control unit 32 determines whether a terminate registration command which terminates the information registration processing has been acquired or not. When the result of the determination at S150 is that a terminate registration command has not been acquired (S150:NO), the control unit 32 returns to S110 of the information registration processing.

Conversely, when the result of the determination at S150 is that a terminate registration command has been acquired (S150:YES), the control unit 32 terminates the information registration processing. Further, the terminate registration command may be acquired when terminating the information display processing explained below, and may be acquired through the input reception unit 44.

In other words, in the information registration processing, the control unit 32 treats any information acquired from each device 20 which represents a specified condition as medical support information, and associates the medical support information with an acquired position and absolute timing and stores the medical support information.

(1-3. Information Display Processing)

Next, an information display processing performed by the control unit 32 will be explained.

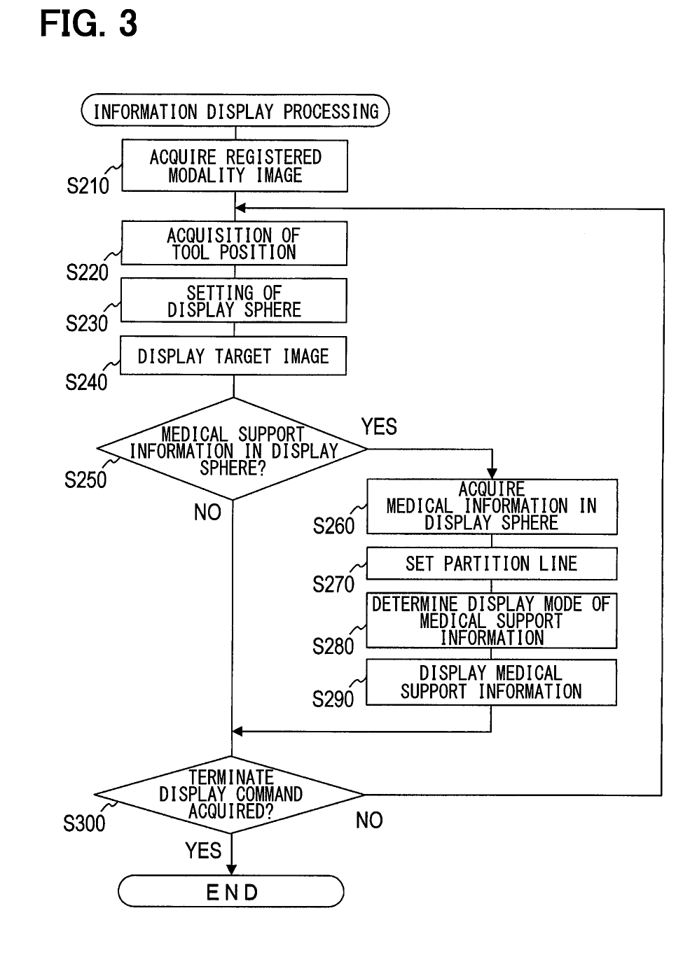

When this information display processing is started, as shown in FIG. 3, first the control unit 32 acquires modality images, which are registered by the registration unit 14 of the medical navigation device 10 with the coordinates for the space where the medical procedure is performed (S210). Next, during the information display processing, the control unit 32 acquires the tool position identified by the position identification unit 12 of the medical navigation device 10 (S220).

Then, the control unit 32 sets a display sphere using the tool position acquired at S220 as a reference point (S230). Here, a display sphere refers to a search region of a predetermined size within the real space where the medical procedure is performed. In one example, the shape of this display sphere may be a sphere. Specifically, at S230, a display sphere defined as a sphere may be set with a center point being the tool position acquired at S220.

Next, during the information display processing, the control unit 32 acquires a target image from within the modality images acquired at S210, and outputs that target image to the display device 50 (S240). Here, a target image refers to an image at a position corresponding to the tool position acquired at S220.

For example, if the modality images include a plurality of tomographic images, the target image may be chosen as the tomographic image taken at the position of the tool position. Further, if the tool position is acquired as a vector from a predetermined reference position to a particular position on the medical tool, the target image may be chosen as an image of a cross section orthogonal to that vector.

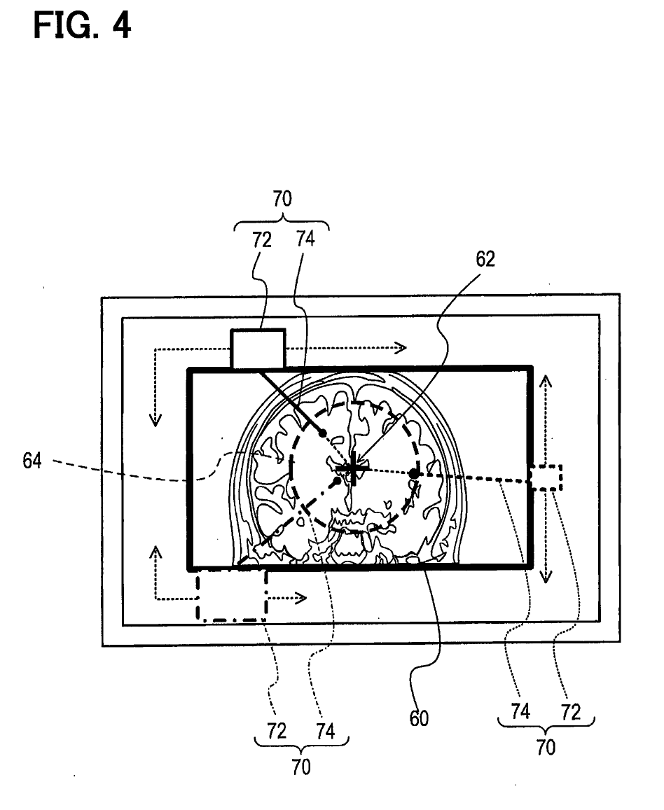

Next, as shown in FIG. 4, the display device 50, which acquired the target mage, displays the target image in an image display region 60 of the display device 50. Here, the image display region 60 refers to a partial region on the display surface of the display device 50, and is the display region of the display device 50 where the target image is displayed.

In the present embodiment, the display of the target image is performed such that a tool position 62 within the target image coincides with the center of the image display region of the display device 50. In FIG. 4, for sake of explaining the display contents, a display sphere 64 is shown, but this display sphere 64 may be not shown as well.

Next, during the information display processing, the control unit 32 determines whether medical support information, which is associated with an acquired position representing being positioned within the display sphere set at S230, exists or not (S250) If the result of the determination at S250 is that medical support information, which is associated with an acquired position representing being positioned within the display sphere, does not exist (S250:NO), the control unit 32 continues the information display processing at S300 described below.

Conversely, if the result of the determination at S250 is that medical support information, which is associated with an acquired position representing being positioned within the display sphere, does exist (S250:YES), the control unit 32 continues the information display processing to S260. At S260, the control unit 32 acquires, from the storage unit 42, all medical support information which is associated with an acquired position representing being positioned within the display sphere.

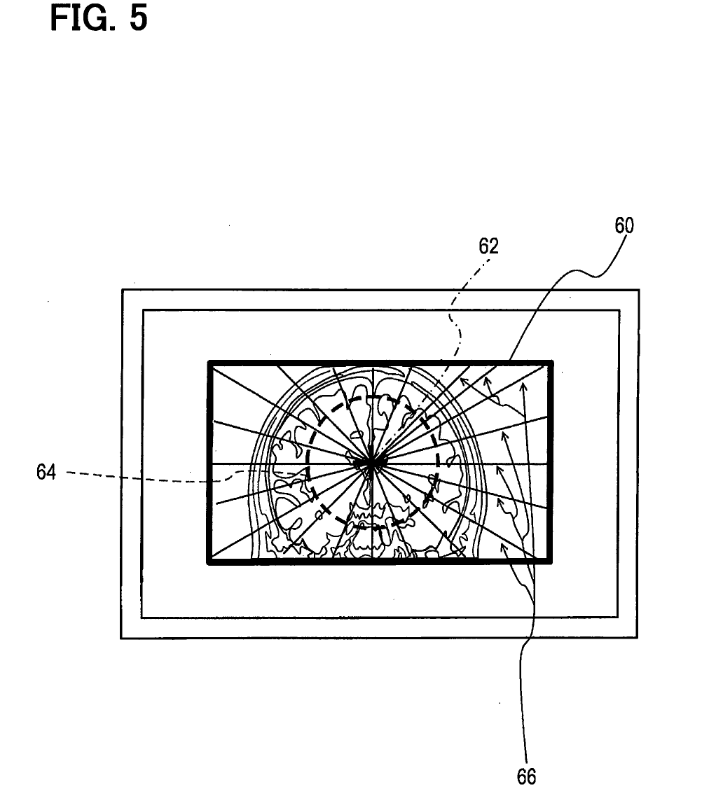

Then, the control unit 32 sets partition lines 66 on the target image displayed in the image display region 60 (S270). Here, the partition lines 66 refer to one or more virtual straight lines. At S270, for example as shown in FIG. 5, each partition line 66 may be set so as to pass through a representative point of the display sphere 64 on the target image, and to be orthogonal to the perimeter of the display sphere 64. Further, the representative point of the display sphere 64 refers to a coordinate which represents the display sphere 64, for example, the center of the display sphere 64.

In FIG. 5, for ease of understanding the display contents, the partition lines 66 are shown on top of the display image. However, in the information display processing, the partition lines 66 do not need to be shown on the target image displayed by the display device 50.

Next, during the information display processing, the control unit 32 performs a mode control that determines a display mode for each medical support information acquired at S260 (S280). Further, the control unit 32 outputs each medical support information acquired at S260 in the display modes determined at S280 to the display device 50 (S290). Then, the display device 50, which acquired each medical support information, performs an information display which associates the acquired medical support information with positions on the target image and displays the acquired medical support information.

Specifically, at S290, the control unit 32 displays an information display frame 70 for each medical support information. Here, an information display frame 70 includes, as shown in FIG. 4, a display frame 72 and a lead line 74. The display frame 72 is a frame in which medical support information is displayed. The lead line 74 is a line that extends from this display frame 72 to a position on the target image corresponding to the acquired position associated with the corresponding medical support information.

Further, at S290, the control unit 32 displays the information display frames 70 on the display device 50 to display the medical support information.

Specifically, at S290, the information display frames 70 are displayed such that each lead line 74 does not overlap with the partition lines 66 set at S270, and such that the lead lines 74 do not overlap with each other. The displaying of the information display frames 70 by the display device 50 is performed according to the display modes determined at S280.

Further, at S290, the control unit 32 may display the information display frames 70 with a different color for each medical support information, or may display the information display frames 70 with a different color for each type of medical support information. In FIG. 4, difference in color in the displayed information display frames 70 is represented by different line times (solid, dashed, one-dot-one-dash, etc.).

Further, at S280, the control unit 32 determines the display modes for the information display frames 70 according to a relative position between the acquired position associated with each corresponding medical support information and the tool position acquired at S220. As one example of a display mode, based on this relative position, the size of the display frame of each information display frame 70 may be changed. Specifically, for example, the size of the display frame of an information display frame 70 may be reduced as a distance from the tool position acquired at S220 to the acquired position associated with each corresponding medical support information increases.

At S280, the control unit 32 further determines the display modes of the information display frames 70 based on a relative position representing whether the acquired position associated with each corresponding medical support information is on the rear side or front side, depth-wise as seen from a practitioner, with respect to the tool position acquired as S220. In this case, when the acquired position associated with each corresponding medical support information is on the front side, depth-wise as seen by the practitioner, with respect to the tool position, then the transparency of the information display frame 70 is increased. Here, transparency refers to the degree to which light passes through, and so as transparency increases, the transmission rate of light increases.

In other words, at S280 and S290, each medical support information which is associated with an acquired position representing being positioned within the display sphere is displayed according to display modes defined based on the relative position between each acquired position and the tool position.

Next, the control unit 32 continues the information display processing to S300.

At S300, the control unit 32 determines whether a terminate display command which terminates the information display processing has been acquired or not. When the result of the determination at S300 is that a terminate display command has not been acquired (S300:NO), the control unit 32 returns to S220 of the information display processing. Conversely, when the result of the determination at S300 is that a terminate display command has been acquired (S300:YES), the control unit 32 terminates the information display processing.

In other words, during the information display processing, the control unit 32 displays a target image corresponding to the tool position, sets a display sphere 64 centered on the tool position, and acquires all medical support information associated with an acquired position representing being located within that display sphere. Then, each medical support information is displayed by the display device 50.

The display of the medical support information is performed by displaying display frames 72 of information display frames 70 around the target image. Further, the display mode of the information display frame 70 is determined based on a relative position between the tool position and each acquired position, and the display of medical support information is performed based on that determined display mode during the information display processing.

Then, during the information display processing, steps S220 to S300 are repeated. During this time, if the tool position changes, then during the information display processing, the target image displayed in the image display region 60 changes along with the change in tool position. Further, during the information display processing, the display of medical support information displayed by the display device 50 changes along with the target image displayed in the image display region 60.

Here, changes in the display of medical support information includes, for example, changing the actual medical support information displayed by the display device 50, changing the manner of display of the medical support information (i.e., the information display frames) shown by the display device 50, changing the display position of the medical support information (i.e., the information display frames) shown by the display device 50, etc. Here, changing the display position of medical support information may be performed such that the lead lines 74 and the partition lines 66 set at S270 do not overlap with each other, and such that each lead line 74 does not overlap with other lead lines 74.

(1-4. Effects of Embodiment)

(1-4a) As explained above, according to the information display processing, at least one medical support information (i.e., one piece of medical support information) may be associated with a position on a target image and displayed. For this reason, according to the information display processing, the corresponding relationship between at least one medical support information and a target image may be easily recognized by a practitioner.

In addition, the target image displayed by the information display processing is an image of a position corresponding to the tool position. For this reason, according to the information display processing, the positional relationship between the tool position and acquired positions may be easily recognized by a practitioner.

These effects, according to the information display processing, may provide a technique for allowing practitioners to more easily recognize medical support information, in techniques which display medical support information.

(1-4b) According to the information display processing, the information display frames 70 are displayed such that the lead lines 74 and the partition lines 66 do not overlap with each other, and such that each lead line 74 does not overlap with other lead lines 74. Accordingly, during the information display processing, the positions of the acquired positions of each medical support information on the target image may be easily recognized by a practitioner.

(1-4c) Further, according to the information display processing, the size of the display frame 72 of an information display frame 70 may be reduced as a distance from the tool position to the acquired position associated with each corresponding medical support information increases. In other words, according to the information display processing, the size of the display frames may be changed according to relative positions. As a result, according to the information display processing, a relative position between the tool position and the acquired positions corresponding to medical support information may be more easily recognized.

(1-4d) Further, according to the information display processing, when the acquired position associated with each corresponding medical support information is on the front side, depth-wise as seen by the practitioner, with respect to the tool position, then the transparency of the information display frame 70 is increased.

Due to this, according to the information display processing, it is possible for a practitioner to recognize whether the acquired position corresponding to medical support information is on the front side or rear side of the tool position, depth-wise as seen by the practitioner. Further, according to the information display processing, due to the degree of transparency, the distance from the tool position to the acquired position may be recognized by the practitioner.

(1-4e) According to the information display processing, each medical support information may be more easily recognized by a practitioner by displaying the information display frame 70 of each medical support information with different colors.

Further, according to the information display processing, a practitioner may recognize the type of medical support information displayed by the display device 50 by changing the display color of the information display frames based on the type of medical support information.

(1-4f) Further, according to the information display processing, an image which is a cross section orthogonal to the tool position vector is displayed by the display device 50 as the target image. The direction of this vector is the direction of a vector from a reference position to a particular position on the medical tool, and is approximate to the viewing direction of the practitioner.

Accordingly, due to the information display processing, an image which is easy to see for a practitioner may be displayed as the target image, and the status of affected parts of a patient may be more easily recognized by the practitioner.

(2. Other Embodiments)

Above, embodiments of the present disclosure are described, but the present disclosure is not limited to the above embodiments, and a variety of embodiments which do not depart from the gist of the present disclosure are contemplated.

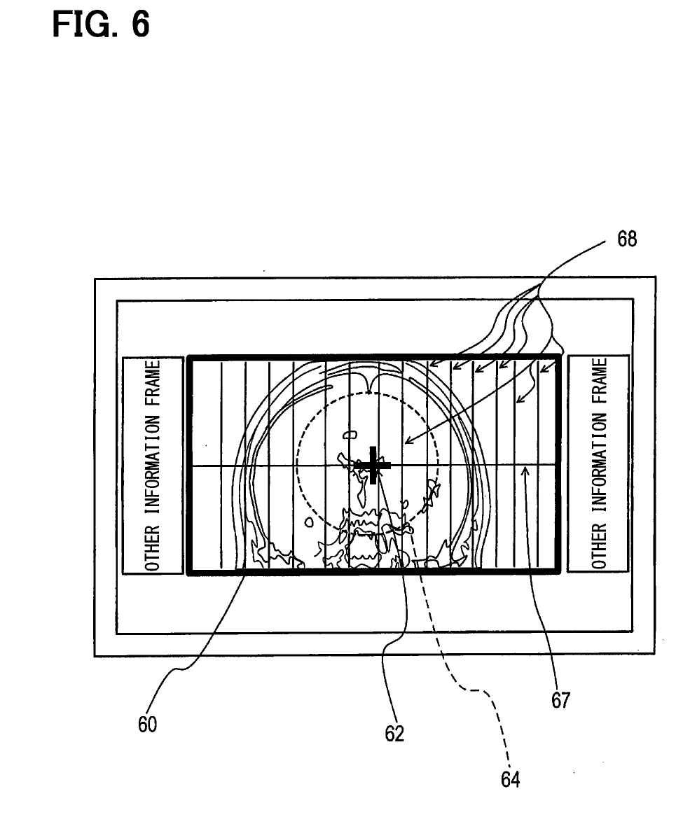

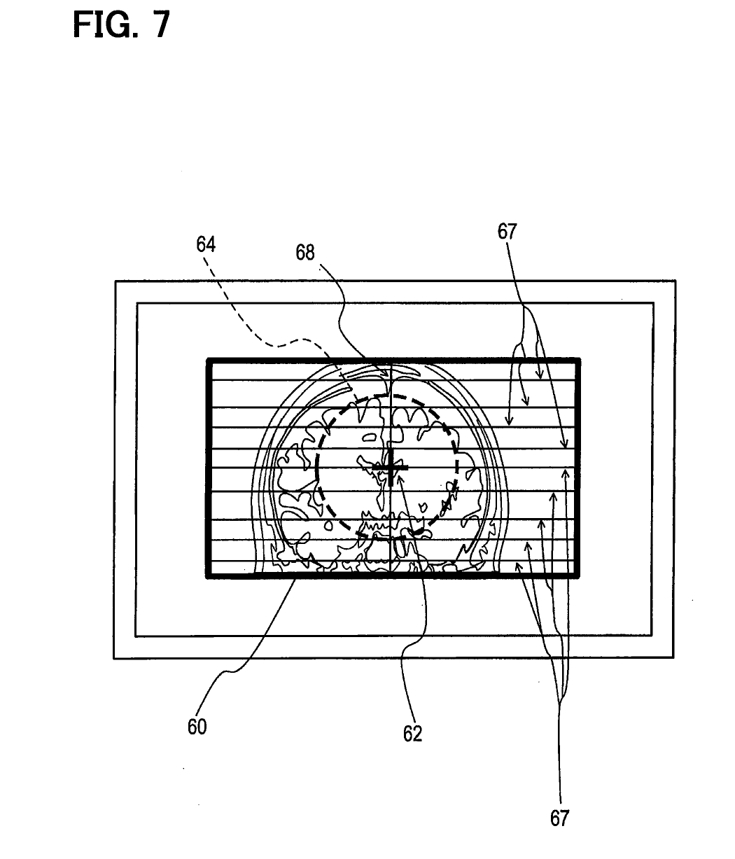

(2.1) In the information display processing of the above embodiments, at S270, the partition lines 66 are set such that each partition line 66 passes through the representative point of the display sphere 64 and is orthogonal to the perimeter of the display sphere 64. However, this setting method for the partition lines 66 is not limited to this. At S270 of the information display processing, as shown in FIGS. 6 and 7, partition lines may be set so as to be horizontal or vertical with respect to the target image.

In this case, as shown in FIG. 6, one partition line 67 may be set so as to be parallel to the horizontal axis of the target image, while a plurality of partition lines 68 may be set to be orthogonal to the horizontal axis of the target image. Further, as shown in FIG. 7, one partition line 68 may be set so as to be parallel to the vertical axis of the target image, while a plurality of partition lines 67 may be set to be orthogonal to the vertical axis of the target image.

In FIGS. 6 and 7, for ease of understanding of the displayed contents, the partition lines 67, 68 are displayed on the target image, but in the information display processing, the partition lines 67, 68 may be not displayed on the target image displayed by the display device 50 instead.

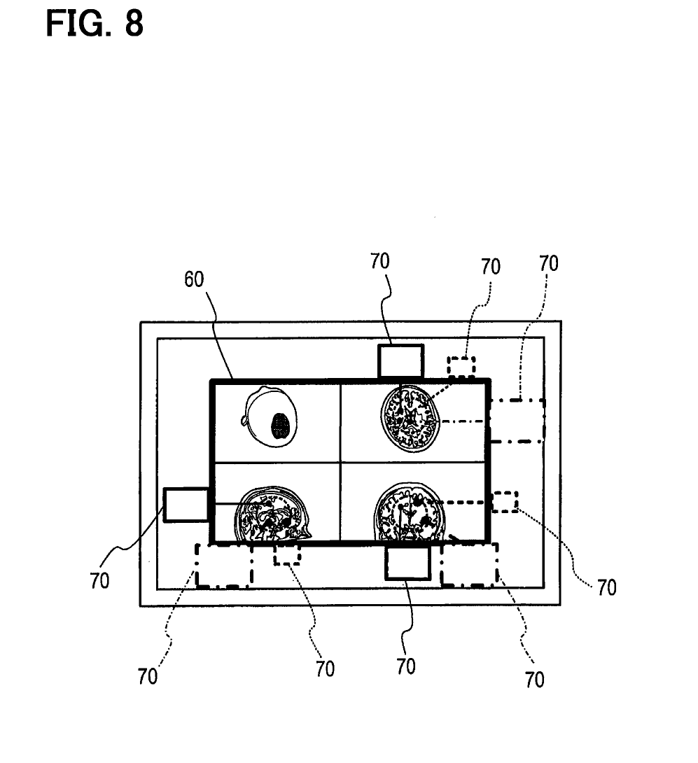

(2.2) According to the information display processing of the above embodiments, at S240, one cross section image from modality images is displayed as the target image. However, the target image displayed at S240 of the information display processing is not limited to this. For example, as shown in FIG. 8, each of a body axis cross section, a sagittal section, a coronal section, and a perspective image may be displayed as target images. In this case, as shown in FIG. 8, each cross section image is displayed in the image display region 60, and further, information display frames 70 may be displayed for each cross section image.

Further, at S240 of the information display processing, any one of a body axis cross section, a sagittal section, a coronal section, or a perspective image may be displayed as a target image instead, and other images of the affect parts of a patient may be displayed instead as well.

In other words, at S240 of the information display processing, images at different cross sections may be displayed by the display device 50 as target images.

(2.3) Further, according to the above embodiments, at S240, as one exemplary mode control, the size of the display frame 72 of each information display frame 70 is changed according to a relative position between the acquired position associated with each corresponding medical support information and the tool position acquired at S220. However, the mode control is not limited to this.

For example, the mode control may be performed by changing the color of the information display frames 70 based on the relative position between the acquired position associated with each corresponding medical support information and the tool position acquired at S220. Further, the mode control may be performed by a combination of changing the size of the display frame 72 of each information display frame 70 and the color of the information display frames 70 according to the relative position.

(2.4) Further, in the above embodiments, when a medical microscope is used as the medical tool, at S120 of the information registration processing and at S220 of the information display processing, the focal position of the medical microscope may be acquired as the tool position.

In this regard, when the focal position of the medical microscope is acquired as the tool position, an image corresponding to that focal position may be displayed as the target image, and an image that coincides with a position matching the viewpoint of a practitioner may be displayed as the target image. As a result, according to the medical support system, the target image may be easily recognized, and a relationship between medical support information and the target image may be more easily recognized.

(2.5) The medical support system 1 of the above embodiments is described with a structure where the medical navigation device 10 and the display control device 30 are separate, but the structure of the medical support system 1 is not limited to this. For example, in the medical support system 1, the medical navigation device 10 may include the display control device 30. In this case, the display control device 30 may also include the functionality of the position identification unit 12 and the registration unit 14.

Further, the device which includes the display control device 30 is not limited to the medical navigation device 10, and, for example, the display control device 30 may be included in a neural function monitoring device or a bio monitoring device.

Further, the display control device 30 may directly acquire the modality images taken by the imaging device 3.

(2.6)

In the above embodiments, a portion or all of the functions performed by the control unit 32 may be implemented in hardware by, for example, a plurality of ICs or the like.

(2.7)

In the above embodiments, programs are stored in the ROM 34, but the storage medium for storing programs is not limited to this. For example, programs may be stored in non-transitory tangible storage media such as semiconductor memory.

(2.8) Further, the control unit 32 executes programs stored on non-transitory tangible storage medium. By executing these programs, methods corresponding to the programs are implemented.

(2.9) Embodiments which omit a part of the structure of the above embodiments are also embodiments of the present disclosure, Further, embodiments from suitably combining the above embodiments or modifications are also embodiments of the present disclosure. Further, all embodiments covered by the technical ideas defined by the expressions recited in the scope of the patent claims are also embodiments of the present disclosure.

(3. Corresponding Relationships)

Functions from performing S210 of the information display processing correspond to an image acquisition unit. Functions from performing S220 correspond to a position acquisition unit. Functions from performing S240 correspond to an image displaying unit. Functions from performing S250, S260 correspond to an information acquisition unit. Functions from performing S270 to S290 correspond to an information displaying unit.

Further, functions from performing S230 correspond to a setting unit. Functions from performing S270 correspond to a line setting unit. Functions from performing S290 correspond to a display performing unit.

What is claimed is:

1. A display method for a display control device to display with a display device at least one medical support information related to supporting a medical practice, comprising:
acquiring modality images including at least one image capturing parts including an affected area of a patient;
repeatedly acquiring a tool position, the tool position being a current position of a medical tool used in the medical practice;
displaying with the display device a target image, the target image being the image among the acquired modality images at a position corresponding to the acquired tool position;
acquiring, as the at least one medical support information, information from a medical practice device other than the medical tool whose tool position is acquired, the at least one medical support information being associated with an acquired position that represents the position of the medical tool at a time when the medical support information was acquired; and
performing an information display by associating, based on the acquired position associated with the acquired at least one medical support information, the medical support information with a position on the target image displayed by the display device and displaying the medical support information.

2. A display control device that displays with a display device at least one medical support information related to supporting a medical practice, comprising:
an image acquisition unit that acquires modality images including at least one image capturing parts including an affected area of a patient;
a position acquisition unit that repeatedly acquires a tool position, the tool position being a current position of a medical tool used in the medical practice;
an image displaying unit that displays with the display device a target image, the target image being the image among the modality images acquired by the image acquisition unit at a position corresponding to the tool position acquired by the position acquisition unit;
an information acquisition unit that acquires, as the at least one medical support information, information from a medical practice device other than the medical tool whose tool position is acquired, the at least one medical support information being associated with an acquired position that represents the position of the medical tool at a time when the medical support information was acquired; and
an information displaying unit that performs an information display by associating, based on the acquired position associated with the acquired at least one medical support information, the medical support information with a position on the target image displayed on the display device by the image displaying unit and that displays the medical support information.

3. The display control device of claim 2, further comprising:
a setting unit that sets a search region which is a region specified with the tool position acquired by the position acquisition unit as a reference point, wherein
the information acquisition unit acquires the at least one medical support information associated with the acquired position that represents being position within the search region set by the setting unit.

4. The display control device of claim 2, wherein
the information displaying unit includes
a line setting unit that sets partition lines on the target image, the partition lines being at least one virtual straight line, and
a display performing unit (30, S290) that performs the information display by displaying
the at least one medical support information, and
a lead line that extends from each of the least one medical support information to a position on the target image corresponding to the acquired position associated with the medical support information, and
the display performing unit performs the information display such that the partition lines set by the line setting unit and the lead line do not overlap with each other, and such that the lead line does not overlap with other lead lines.

5. The display control device of claim 4, wherein
the line setting unit sets the partition lines so as to pass through a representative point of the search region set by the setting unit.

6. The display control device of claim 4, wherein
the line setting unit sets the partition lines so as to be horizontal or vertical with respect to the target image.

7. The display control device of claim 4, wherein
the display performing unit performs the information display by displaying an information display frame for each of the at least one medical support information, the information display frame including
a display frame in which a corresponding one of the at least one medical support information is displayed, and
a lead line that extends from the display frame.

8. The display control device of claim 7, wherein
the display performing unit performs the information display by displaying each of the at least one medical support information with a different color.

9. The display control device of claim 7, wherein
the display performing unit performs the information display by displaying with a different color for each type of the at least one medical support information.

10. The display control device of claim 7, wherein
the display performing unit performs a mode control as the information display, the mode control including controlling a display mode of the information display frame based on a relative position between the acquired position and the tool position acquired by the position acquisition unit.

11. The display control device of claim 10, wherein
the display performing unit performs the mode control by changing a color of the information display frame based on the relative position between the acquired position and the tool position acquired by the position acquisition unit.

12. The display control device of claim 10, wherein
the display performing unit performs the mode control by changing a size of the display frame based on the relative position between the acquired position and the tool position acquired by the position acquisition unit.

13. The display control device of claim 12, wherein
the display performing unit performs the mode control by reducing the size of the display frame as a distance between the acquired position and the tool position acquired by the position acquisition unit increases.

14. The display control device of claim 10, wherein
the display performing unit performs the mode control with the relative position being whether the acquired position is on a rear side or a front side of the tool position, depth-wise as seen by a practitioner.

15. The display control device of claim 14, wherein
the display performing unit performs the mode control by increasing a transparency of the information display frame when the acquired position is in front of the tool position, depth-wise as seen by a practitioner.

16. The display control device of claim 2, wherein
the position acquisition unit acquires, as the tool position, a vector from a predetermined reference position to a particular position on the medical tool, and
the image displaying unit displays, as the target image on the display device, the image of a cross section orthogonal to the vector acquired as the tool position.

17. The display control device of claim 2, wherein
the image displaying unit displays, as the target image on the display device, images at different cross sections as each other.
